(19) United States
(12) Patent Application Publication
(10) Pub. No.: US 2021/0202076 A1
(43) Pub. Date: Jul. 1, 2021

(54) ANALYZING CLINICAL PATHWAYS

(71) Applicant: KONINKLIJKE PHILIPS N.V., EINDHOVEN (NL)

(72) Inventor: PATRICK CHEUNG, Eindhoven (NL)

(21) Appl. No.: 16/754,399

(22) PCT Filed: Oct. 31, 2018
(86) PCT No.: PCT/EP2018/079775

§ 371 Date: Apr. 8, 2020

Related U.S. Application Data
(60) Provisional application No. 62/583,150, filed on Nov. 8, 2017.

(51) Int. Cl. G16H 40/20; G16H 10/65

(52) U.S. Cl. CPC G16H 40/20; G16H 10/65

(57) ABSTRACT

Methods and systems for studying clinical pathways. Methods and systems described herein implement a two-stage clustering approach for learning clinical pathways. A first clustering procedure is executed on patient data to sort the data into clusters based on clinical path structure. Then a second clustering procedure is executed on the data based on the combination of clinical path structure and relevant contextual variables that affect clinical pathways.

### TECHNICAL FIELD

Embodiments described herein generally relate to systems and methods for analyzing patient data and, more particularly but not exclusively, to systems and methods for analyzing patient data to study clinical pathways.

### BACKGROUND

The adoption of electronic medical records (EMRs) in hospitals and other healthcare institutions provides an opportunity to develop data-driven methods to study clinical pathways in practice. Clinical pathways are defined as structured multidisciplinary plans that detail steps in the care of patients. Knowledge of clinical pathways can support the translation of clinical guidelines into local protocols and clinical practice.

For example, data-driven methods to study clinical pathways can help identify clinical activities that are commonly performed by physicians and other medical personnel for patients with certain diagnoses. Clinical pathway knowledge can also help reduce undesired practice variability and provide clinical decision support. Accordingly, studying clinical pathways can optimize patient outcomes and maximize clinical efficiency by improving resource utilization, reducing length of stay, and reducing hospital costs.

Existing techniques for analyzing clinical pathways focus on mining the order and temporal information of clinical activities for patients with similar diagnoses. These clinical activities may include diagnostic and treatment activities such as blood tests, infrared treatments, or the like. However, two concerns are raised to such approach. First of all, it is difficult, if not impossible, to identify only a small number of subgroups of clinical pathways by grouping clinical pathways with similar clinical activities (i.e. path structures) due to the high variations in the clinical process. Second, patients with different demographics, previous test results, chronic illnesses, medications, or the like, may take different pathways in order to achieve desired outcome. Such variations should be taken into consideration during the identification of clinical pathway subgroups.

Therefore, knowledge about clinical pathways may be of limited use if it does not consider patient context and outcomes. However, identifying contextual variables may be difficult as not all contextual variables affect clinical pathways. Moreover, many contextual variables are unknown in advance.

A need exists, therefore, for systems and methods for studying clinical pathways that overcomes the above disadvantages.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a method for studying clinical pathways. The method includes receiving patient data records using an interface; extracting, using an extraction module, a plurality of clinical pathways from the patient data records; executing, using a clustering module, a first clustering procedure to sort the plurality of clinical pathways into a plurality of clusters based on the structure of the pathways; extracting, using the extraction module, contextual variable data from the patient data records; identifying at least one contextual variable from the extracted contextual variable data; and executing, using the clustering module, a second clustering procedure to sort the plurality of clinical pathways into a second plurality of clusters based on at least one identified contextual variable and the structure of the pathways.

In some embodiments, identifying the at least one contextual variable includes: comparing statistical distributions of each of a plurality of contextual variables among the plurality of clusters, and selecting at least one contextual variable with the highest distribution discrepancy.

In some embodiments, the method further includes identifying structure similarity between two clinical pathways by comparing clinical events between two pathways and calculating structure similarity using the maximum number of ordered events that the two pathways have in common.

In some embodiments, the at least one contextual variable is selected from the group consisting of demographics, social history, prior hospitalizations, previous test results, diagnoses, and medical interventions.

In some embodiments executing the second clustering procedure, includes calculating a composite similarity function based on path structure similarity and contextual similarity between two clinical pathways.

In some embodiments, the method further includes supplying using the interface, analytical results after executing the second clustering procedure, wherein the analytical results include data selected from the group consisting of common clinical pathways, demographics, length of patient stay, and healthcare cost.

According to another aspect, embodiments relate to a system for studying clinical pathways. The system includes an interface configured to receive patient data records; a memory; and a processor executing instructions stored on the memory to provide: an extraction module configured to: extract a plurality of clinical pathways from the patient data records, and extract contextual variable data from the patient data records; and a clustering module configured to: execute a first clustering procedure to sort the plurality of clinical pathways into a plurality of clusters based on the structure of the pathways, wherein the extraction module is further configured to identify at least one contextual variable from the extracted contextual variable data, and execute a second clustering procedure to sort the plurality of clinical pathways into a second plurality of clusters based on at least one identified contextual variable and the structure of the pathways.

In some embodiments, the extraction module identifies the at least one contextual variable by: comparing statistical distributions of each of a plurality of contextual variables among the plurality of clusters, and selecting at least one contextual variable with the highest distribution discrepancy.

In some embodiments, the extraction module is further configured to identify structure similarity between two clinical pathways by comparing clinical events between two pathways and calculating structure similarity using the maximum number of ordered events that the two pathways have in common.

In some embodiments, the at least one contextual variable is selected from the group consisting of demographics, social history, prior hospitalizations, previous test results, diagnoses, and medical interventions.

In some embodiments, the clustering module executes the second clustering procedure by calculating a composite similarity function based on path structure similarity and contextual similarity between two clinical pathways.

In some embodiments, the interface is configured to supply analytical results after executing the second clustering procedure, wherein the analytical results include data selected from the group consisting of common clinical pathways, demographics, length of patient stay, and healthcare cost.

According to yet another aspect, embodiments relate to a computer readable medium containing computer-executable instructions for studying clinical pathways. The medium includes computer-executable instructions for receiving patient data records using an interface; computer-executable instructions for extracting, using an extraction module, a plurality of clinical pathways from the patient data records; computer-executable instructions for executing, using a clustering module, a first clustering procedure to sort the plurality of clinical pathways into a plurality of clusters based on the structure of the pathways; computer-executable instructions for extracting, using the extraction module, contextual variable data from the patient data records; computer-executable instructions for identifying at least one contextual variable from the extracted contextual variable data; and computer-executable instructions for executing, using the clustering module, a second clustering procedure to sort the plurality of clinical pathways into a second plurality of clusters based on at least one identified contextual variable and the structure of the pathways.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a system for studying clinical pathways in accordance with one embodiment;

FIG. 2 depicts the workflow of the various components of FIG. 1 for optimizing clinical pathways analysis in accordance with one embodiment;

FIG. 3 illustrates a clinical pathway in accordance with one embodiment;

FIGS. 4A, 4B, 4C, 4D and 4E illustrate a two-stage clustering procedure in accordance with one embodiment; and

FIG. 5 depicts a flowchart of a method for studying clinical pathways in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The systems and methods in accordance with various embodiments described herein concern novel techniques to study and optimize clinical pathways by considering both clinical path structure and relevant contextual information. These approaches use clinical path structure as the principal feature to select a subset of contextual variables based on their statistical distributions. This takes into account or is otherwise based on the fact that patients who share the same context and follow similar clinical paths should achieve the same outcome.

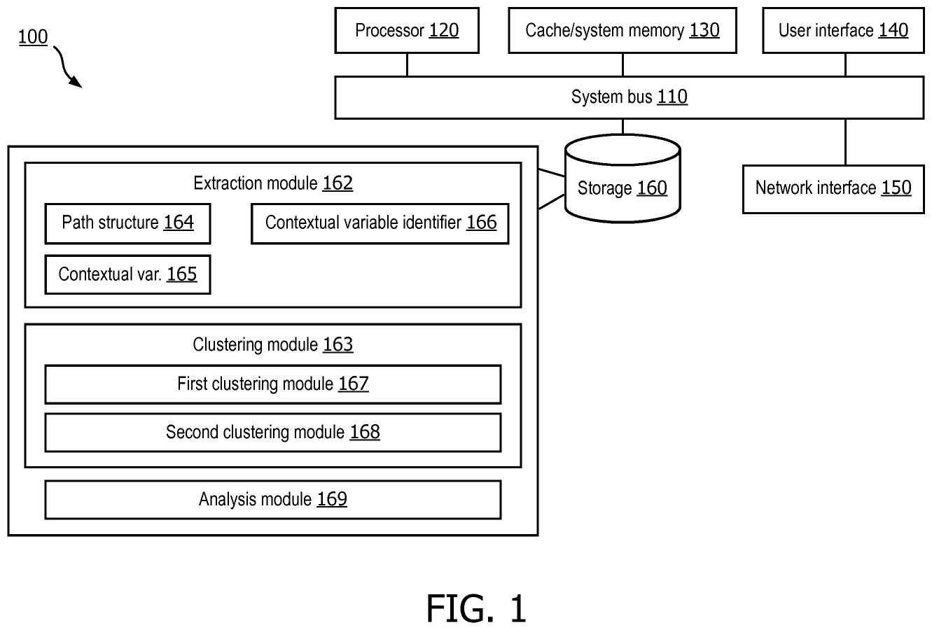

FIG. 1 illustrates a system 100 for optimizing clinical pathways analysis in accordance with one embodiment. The system 100 includes a processor 120, memory 130, a user interface 140, a network interface 150, and storage 160 interconnected via one or more system buses 110. It will be understood that FIG. 1 constitutes, in some respects, an abstraction and that the actual organization of the system 100 and the components thereof may differ from what is illustrated.

The processor 120 may be any hardware device capable of executing instructions stored on memory 130, on storage 160, or otherwise capable of processing data. As such, the processor 120 may include a microprocessor, field programmable gate array (FPGA), application-specific integrated circuit (ASIC), or other similar devices.

The memory 130 may include various memories such as, for example L1, L2, or L3 cache or system memory. As such, the memory 130 may include static random access memory (SRAM), dynamic RAM (DRAM), flash memory, read only memory (ROM), or other similar memory devices and configurations.

The user interface 140 may include one or more devices for enabling communication with a user such as medical personnel. For example, the user interface 140 may include a display, a mouse, and a keyboard for receiving user commands In some embodiments, the user interface 140 may include a command line interface or graphical user interface that may be presented to a remote terminal via the network interface 150. The user interface 140 may execute on a user device such as a PC, laptop, tablet, mobile device, or the like.

The network interface 150 may include one or more devices for enabling communication with other hardware devices. For example, the network interface 150 may include a network interface card (NIC) configured to communicate according to the Ethernet protocol. Additionally, the network interface 150 may implement a TCP/IP stack for communication according to the TCP/IP protocols. Various alternative or additional hardware or configurations for the network interface 150 will be apparent.

The network interface 150 may be in operable communication with one or more databases. These databases may store data regarding patients such as EMRs that contain clinical pathway data.

The storage 160 may include one or more machine-readable storage media such as read-only memory (ROM), random-access memory (RAM), magnetic disk storage media, optical storage media, flash-memory devices, or similar storage media. In various embodiments, the storage 160 may store instructions for execution by the processor 120 or data upon which the processor 120 may operate.

For example, the storage 160 may include an extraction module 162 for extracting and identifying certain information and a clustering module 163 for executing various clustering procedures. The extraction module 162 may be configured with a path structure module 164, a contextual variable module 165, and a contextual variable identifier module 166.

The clustering module 163 may be configured with a first clustering module 167 to execute a first clustering procedure and a second clustering module 168 to execute a second clustering procedure. The storage may 160 may also include an analysis module 169. It is noted, however, that the tasks carried out by the various modules are or involve processing functions and, as such, the various modules may be configured with or otherwise as part of the processor 120.

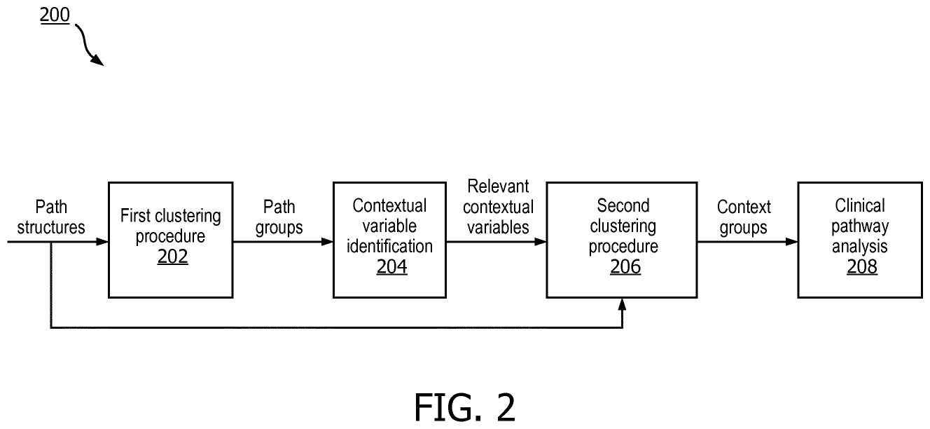

FIG. 2 illustrates a workflow 200 of the various components of the system 100 of FIG. 1. The extraction module 162 may first gather or otherwise receive patient EMRs from one or more databases or data sources. A user such as a clinician or other type of medical personnel may select certain EMRs based on certain criteria.

For example, the user may segment the records by the study of interest such as patient outcomes or claims. The user may use any appropriate input devices configured with the user interface 140 to set various segmenting parameters.

The path structure module 164 may extract a plurality of clinical pathways (i.e., path structures) from the EMRs based on the defined criteria. In the context of the present application, the term “clinical pathways” or “path structures” may refer to the series of steps or actions undergone by a patient. These may include, for example, tests performed on the patient, diagnoses made regarding the patient, results of tests performed on the patient, or the like.

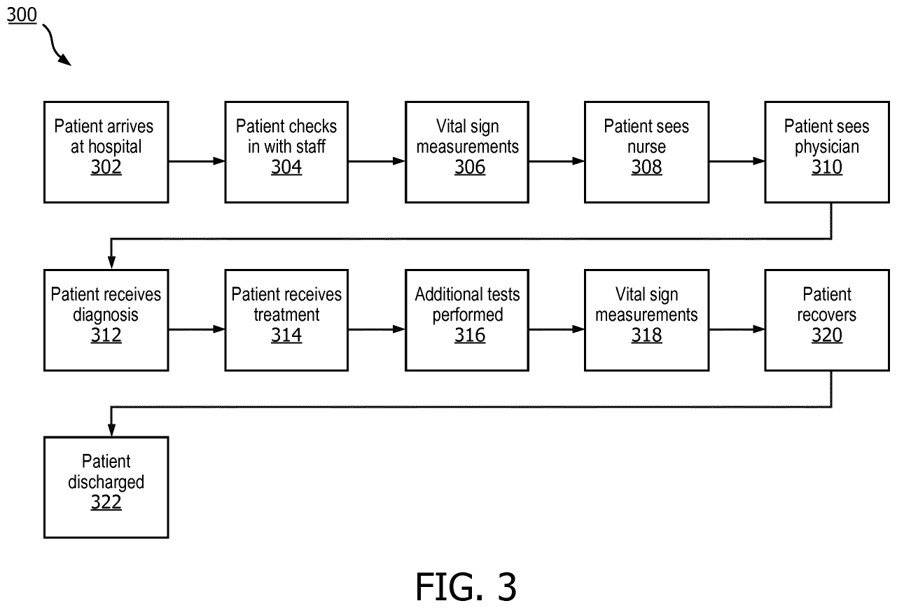

FIG. 3 illustrates an exemplary clinical pathway 300 that illustrates a series of events related to a patient's visit to a hospital (or other type of healthcare institution). First, a patient may arrive at the hospital (S302) and check in with the hospital staff (S304). Then, a patient may be moved to a room or other designated area within the hospital to have their vital signs measured (S306). Afterwards or concurrently with (S306) the patient may visit and discuss with a nurse (S308).

Then, the patient may see a physician or other type of medical personnel (S310) to further describe their ailments. After the physician reviews the patient's complaints and measurements the physician may provide a diagnosis (S312). The diagnosis may call for a series of medical treatments (S314) and additional tests (S316). After tests are performed, or throughout the patient's visit to the hospital, the patient's vital signs may continuously be measured (S318). Assuming the tests and treatments were successful, the patient then recovers (S320) and eventually is discharged from the hospital (S322).

Referring back to FIG. 2, the first clustering module 167 may execute a first clustering procedure to sort the plurality of clinical pathways into a plurality of clusters based on their structure (S202). For example, the first clustering module 167 may sort pathways based on path similarity. In some embodiments the first clustering module 167 may identify pathways with the longest common ordered events (i.e., subsequences) and sort them into clusters. That is, the first cluster module 167 may find pathways that have the same common steps such as steps 312, 314, 316, and 318 of FIG. 3 (i.e., in which the patient received the same diagnosis and the same treatment in steps 312 and 314, respectively).

Next, the contextual variable module 165 may extract contextual variable data from the EMRs (S204). In the context of the present application, the terms “contextual variable data” or “contextual data” may refer to data related to patients such as their demographics, social history, prior hospitalizations, and previous test results, diagnoses, and medical interventions.

Then, the contextual variable identifier module 166 may identify relevant contextual variables from those extracted by the contextual variable module 165 (S204). To identify which variables are “relevant,” the identifier module 166 may compare statistical distributions of each identified contextual variable among the clusters. For example, the contextual variable identifier module 166 may compare the Kullback-Leibler divergence of certain contextual variables. Variables with large distribution discrepancies are considered to be relevant contextual variables that affect clinical pathways, and may be selected for further analysis.

The second clustering module 168 may then execute a second clustering procedure to sort the plurality of clinical pathways into a second plurality of clusters based on at least one identified contextual variable and the structure of the pathways (S206). During S206, the second clustering module 168 may calculate the weighted sum of the path similarities and contextual similarities.

For example, if the trade-off between path structure and contextual similarities is considered, the second clustering module 168 may assign a weight a to a similarity function representing the path structure similarity and a weight (1−α) to a similarity function representing the contextual similarity between two clinical pathways. In some embodiments, a composite similarity function may comprise a contextual similarity function and a path structure similarity function and may be written as:

sim(x, y)=αx path_sim(x, y)+(1−α)×context_sim(x, y)

where α may be manually chosen and may be between 0 and 1. For example, if a user wanted to heavily emphasize the similarity between paths, the user may select an α closer to 1. If the user wanted to emphasize more on the similarity between contextual variables, the user may select an α closer to 0.

After the second clustering module 168 executes the second clustering procedure, the analysis module 169 or a user may perform clinical analysis on the generated clusters (S208). At this point, the clustering modules 167 and 168 have identified groups of similar patients in terms of both patient context and clinical pathways. The analysis module 169 may then use an appropriate sequential pattern mining technique (e.g., Sequential PAttern Discovery using Equivalence classes (SPADE)) to extract information such as common clinical pathways or to identify anomalies within each cluster (i.e., within each context group).

Referring back to FIG. 1, the user interface 140 may execute a visualization tool to display analytical results. These analytical results may include, for example, common clinical pathways, length of stays for patients, hospital costs, or the like to help medical personnel understand patient care flow and hospital workflow.

For example, the analytical results may reveal that patients above a certain age went through additional tests that were not performed on other patient groups with the same diagnosis. Or it is possible that a certain hospital kept patients in the Emergency Department before transferring them to a general ward for a longer time period than other hospitals. Further studies may recognize problems or shortcomings in the urgency categorization in a particular hospital or healthcare institution, as well as the corrective actions that were taken to reduce patient turnaround time.

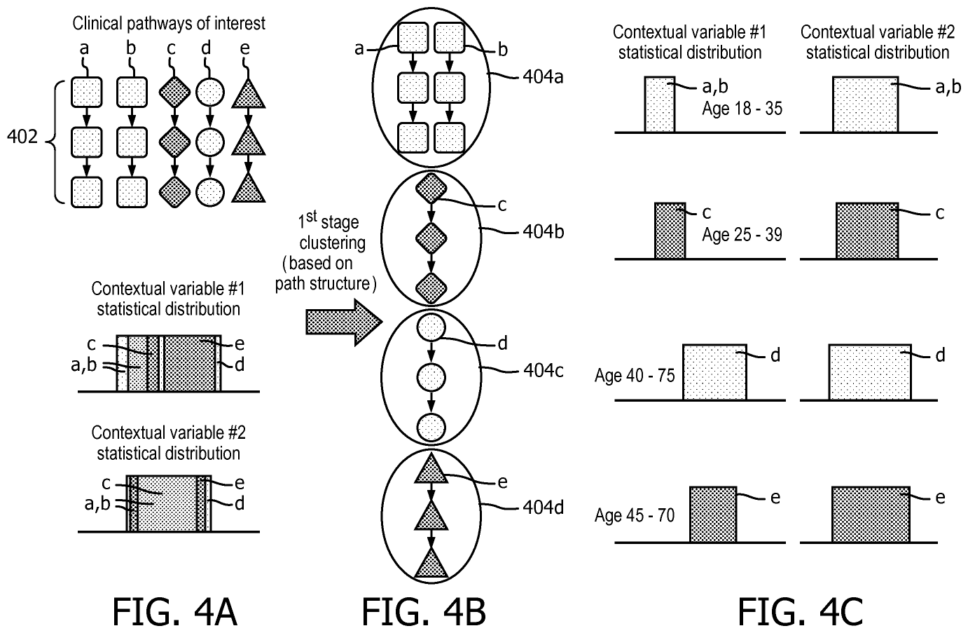

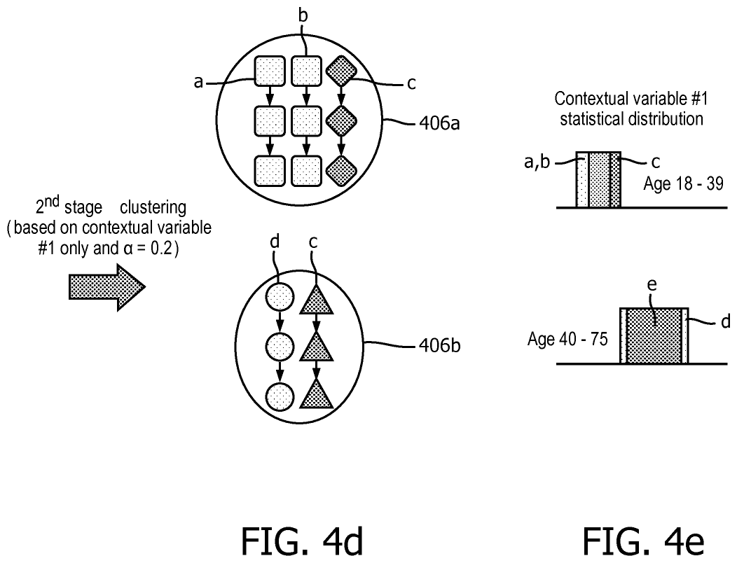

FIGS. 4A-E illustrate the two-stage clustering procedure in accordance with one embodiment. FIG. 4A shows a plurality of clinical pathways 402 (labeled a, b, c, d, and e), as well as the distributions of contextual variable 1 (e.g., patient age) and contextual variable 2 among the patients associated with paths a-e.

After the first clustering module 167 executes the first clustering procedure, the plurality of paths 402 are sorted into clusters 404a, 404b, 404c, and 404d as shown in FIG. 4B. That is, the clusters 404a, 404b, 404c, and 404d sort the plurality of paths a-e based on their path structure. For example, cluster 404a includes paths a and b, which may have a similar path structure.

FIG. 4C illustrates the distributions of contextual variables 1 and 2. Contextual variable 1 (e.g., age) is more relevant than contextual variable 2 (which may relate to some other patient characteristic) as contextual variable 1 has a larger distribution discrepancy among the clusters. For example, patients associated with paths a and b may be 18-35 years old, the patient associated with path c may be 25-39 years old, the patient associated with path d may be 40-75 years old, and the patient associated with path e may be 45-70 years old.

FIGS. 4D and 4E illustrate clusters 406a and 406b after the second clustering module 168 executes the second clustering procedure. Specifically, FIGS. 4D and E represent the generated clusters based on the contextual variable 1 and α=0.2. That is, the composite similarity function discussed above focuses 20% on the similarity between the patients with respect to path structures and 80% on the similarity between the patients with respect to contextual variable 1.

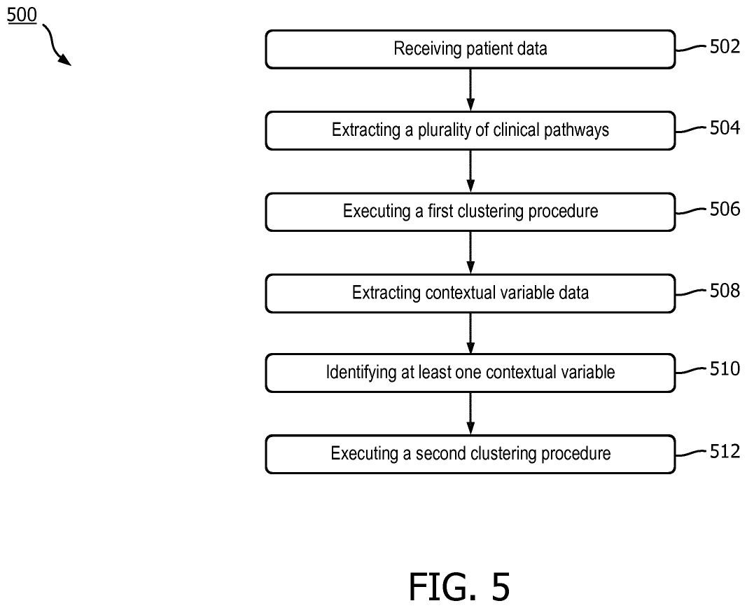

FIG. 5 depicts a flowchart of a method 500 for studying clinical pathways in accordance with one embodiment. Step 502 involves receiving patient data records using an interface. This patient data may include patient EMRs obtained from a hospital database, for example.

Step 504 involves extracting, using an extraction module, a plurality of clinical pathways from the patient data records. The extraction module may be similar to the extraction module 162 of FIG. 1, for example. As discussed above, clinical pathways refer to the steps or actions taken by a patient at a healthcare institution. These include received diagnoses as well as tests or actions performed on the patients.

Step 506 involves executing, using a clustering module, a first clustering procedure to sort the plurality of clinical pathways into a plurality of clusters based on the structure of the pathways. The clustering module may be similar to the first clustering module 163 of FIG. 1, for example. In some embodiments, the clustering module may sort the pathways based on those that have the longest common ordered events between two pathways.

Step 508 involves extracting, using the extraction module, contextual variable data from the patient data records. In some embodiments, contextual variables may include at least one of demographics, social history, prior hospitalizations, and previous test results, diagnoses, and medical interventions.

Step 510 involves identifying at least one contextual variable based on the extracted contextual variable data from at least one cluster. This step may be performed by the contextual variable identifier module 166 of FIG. 1, for example. The contextual variable identifier module 166 may identify a relevant variable by comparing statistical distributions of each of a plurality of contextual variables among the plurality of clusters and then selecting at least one contextual variable with the highest distribution discrepancy.

Step 512 involves executing, using the clustering module, a second clustering procedure to sort the plurality of clinical pathways into a second plurality of clusters based on at least one identified contextual variable and the structure of the pathways. Specifically, the second clustering module 168 of FIG. 1 may execute the second clustering procedure.

In some embodiments, the second clustering module may execute a function that calculates a composite similarity function based on structure similarity and the contextual similarity between two paths. Accordingly, the groups or clusters formed may vary by considering either the contextual variables or path structures more heavily than the other.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for studying clinical pathways, the method comprising:
receiving patient data records using an interface;
extracting, using an extraction module, a plurality of clinical pathways from the patient data records;
executing, using a clustering module, a first clustering procedure to sort the plurality of clinical pathways into a plurality of clusters based on the structure of the pathways;
extracting, using the extraction module, contextual variable data from the patient data records;
identifying at least one contextual variable from the extracted contextual variable data; and
executing, using the clustering module, a second clustering procedure to sort the plurality of clinical pathways into a second plurality of clusters based on at least one identified contextual variable and the structure of the pathways.

2. The method of claim 1 wherein identifying the at least one contextual variable includes:
comparing statistical distributions of each of a plurality of contextual variables among the plurality of clusters, and
selecting at least one contextual variable with the highest distribution discrepancy.

3. The method of claim 1 further comprising identifying structure similarity between two clinical pathways by comparing clinical events between two pathways and calculating structure similarity using the maximum number of ordered events that the two pathways have in common.

4. The method of claim 1 wherein the at least one contextual variable is selected from the group consisting of demographics, social history, prior hospitalizations, previous test results, diagnoses, and medical interventions.

5. The method of claim 1 wherein executing the second clustering procedure includes calculating a composite similarity function based on path structure similarity and the contextual similarity between two clinical pathways.

6. The method of claim 1 further comprising supplying, using the interface, analytical results after executing the second clustering procedure, wherein the analytical results include data selected from the group consisting of common clinical pathways, demographics, length of patient stay, and healthcare cost.

7. A system for studying clinical pathways, the system comprising:
an interface configured to receive patient data records;
a memory; and
a processor executing instructions stored on the memory to provide:
an extraction module configured to:
extract a plurality of clinical pathways from the patient data records, and
extract contextual variable data from the patient data records; and
a clustering module configured to:
execute a first clustering procedure to sort the plurality of clinical pathways into a plurality of clusters based on the structure of the pathways, wherein the extraction module is further configured to identify at least one contextual variable from the extracted contextual variable data, and
execute a second clustering procedure to sort the plurality of clinical pathways into a second plurality of clusters based on at least one identified contextual variable and the structure of the pathways.

8. The system of claim 7 wherein the extraction module identifies the at least one contextual variable by:
comparing statistical distributions of each of a plurality of contextual variables among the plurality of clusters, and
selecting at least one contextual variable with the highest distribution discrepancy.

9. The system of claim 7 wherein the extraction module is further configured to identify structure similarity between two clinical pathways by comparing clinical events between two pathways and calculating structure similarity using the maximum number of ordered events that the two pathways have in common.

10. The system of claim 7 wherein the at least one contextual variable is selected from the group consisting of demographics, social history, prior hospitalizations, previous test results, diagnoses, and medical interventions.

11. The system of claim 7 wherein the clustering module executes the second clustering procedure by calculating a composite similarity function based on path structure similarity and contextual similarity between two clinical pathways.

12. The system of claim 7 wherein the interface is configured to supply analytical results after executing the second clustering procedure, wherein the analytical results include data selected from the group consisting of common clinical pathways, demographics, length of patient stay, and healthcare cost.

13. A computer readable medium containing computer-executable instructions for studying clinical pathways, the medium comprising:
computer-executable instructions for receiving patient data records using an interface;
computer-executable instructions for extracting, using an extraction module, a plurality of clinical pathways from the patient data records;
computer-executable instructions for executing, using a clustering module, a first clustering procedure to sort the plurality of clinical pathways into a plurality of clusters based on the structure of the pathways;
computer-executable instructions for extracting, using the extraction module, contextual variable data from the patient data records;
computer-executable instructions for identifying at least one contextual variable from the extracted contextual variable data; and
computer-executable instructions for executing, using the clustering module, a second clustering procedure to sort the plurality of clinical pathways into a second plurality of clusters based on at least one identified contextual variable and the structure of the pathways.
